(19) United States
(12) Patent Application Publication
(10) Pub. No.: US 2019/0231236 A1
(43) Pub. Date: Aug. 1, 2019

(54) ACCURATE ENZYMATIC SENSING OF SWEAT ANALYTES

(71) Applicants: University Of Cincinnati, Cincinnati, OH (US); Eccrine Systems, Inc., Cincinnati, OH (US)

(72) Inventors: Jason Charles Heikenfeld, Cincinnati, OH (US); Jacob Bertrand, Norwood, OH (US); Michael Charles Brothers, Lebanon, OH (US); Eliot Gomez, Cincinnati, OH (US)

(21) Appl. No.: 16/334,784

(22) PCT Filed: Sep. 21, 2017
(86) PCT No.: PCT/US2017/052651

§ 371 Date: Mar. 20, 2019

Related U.S. Application Data
(60) Provisional application No. 62/397,706, filed on Sep. 21, 2016.

(51) Int. Cl. A61B 5/145; A61B 5/1486; A61B 5/00; A61B 5/053

(52) U.S. Cl. CPC A61B 5/14517; A61B 5/1486; C12Q 1/70; A61B 5/0533; A61B 5/6833

(57) ABSTRACT

A device (200) for sensing a biofluid (18) includes at least one analyte-consuming sensor (220) for measuring at least a first analyte concentration of an analyte in the biofluid (18) and at least one additional component (248). The at least one additional component (248) maintains analyte-consuming sensor (220) measurements within 20% of the first concentration measurement if a biofluid (18) sample flow rate is less than or equal to 2 times a first biofluid (18) sample flow rate measurement as measured by the device (200). A method for sensing a biofluid (18) includes measuring a first analyte concentration of an analyte in the biofluid (18) using an analyte-consuming sensor (220), measuring a first biofluid sample flow rate, and maintaining a subsequent analyte concentration measurement within 20% of the first analyte concentration measurement when a subsequently measured biofluid (18) flow rate is less than or equal to 2 times the first biofluid sample flow rate.

### BACKGROUND OF THE INVENTION

Non-invasive biosensing technologies have enormous potential for applications ranging from athletics, to neonatology, to pharmacological monitoring, to personal digital health, to name a few applications. The sweat ducts can provide a route of access to many of the same biomarkers, chemicals, or solutes that are carried in blood and can provide significant information enabling one to diagnose ailments, health status, toxins, performance, and other physiological attributes even in advance of any physical sign. Sweat has many of the same analytes and analyte concentrations found in blood and interstitial fluid. Interstitial fluid has even more analytes nearer to blood concentrations than sweat does, especially for larger sized and more hydrophilic analytes (e.g., proteins).

If biofluid access through the skin has such significant potential as a sensing paradigm, then why has it not emerged beyond decades-old usage in infant chloride sweat assays for Cystic Fibrosis or in illicit drug monitoring patches? Or, why did past reverse iontophoresis products for interstitial fluid extraction, such as GlucoWatch, fail commercially? Past challenges and failures have been at least partially due to the difficulty of finding ergonomic and acceptable ways to generate the biofluid for sampling (non-invasive, continuous, non-irritating, etc.). Further, past efforts experienced difficulty in obtaining an adequate sample volume for a measurement of analytes in these types of biofluids. Reducing the sample volume is critical for rapid sampling or to allow lower biofluid generation rates (e.g., less reverse iontophoresis current and related stress on skin). However, simply reducing the biofluid volume, especially when using reverse iontophoresis, causes secondary challenges, such as pH changes.

A more detailed background description of biofluid sampling rate is now provided. Assume sweat glands predominantly provide pre-existing pathways for biofluid extraction. Next, with reference to Cunningham, In Vivo Glucose Sensing, 2010, assume a device having a sampling area of 1 cm² is applied to a wearer's wrist. Assuming a sweat gland density of 150/cm² for the wrist, a sensor having a 0.55 cm radius (1.1 cm in diameter) would cover about 1 cm² area, or approximately 150 sweat glands. Next, assume the device applies 3 minutes of reverse iontophoresis at 0.3 mA/cm², and generates 15 to 150 nL of biofluid. Therefore, roughly 5 to 50 nL of biofluid is generated per minute, which is a sample generation rate of roughly 0.03 to 0.3 nL/min/gland. If the fluidic portion of the 1 cm² device is 127 μm thick (the same thickness as the gel used with GlucoWatch), then fluidic volume is 12,700 nL. If that volume were to be completely filled with new biofluid, it would require 282 to 2822 minutes (47 to 4.7 hours), which represents a very slow sampling interval.

Next, consider the effect of the sensing modality on the required sampling interval and the analyte detection range of a device. Whether a sensor consumes the sensed analyte as it is detected, or binds and releases the analyte back into solution, is a primary distinction among sensing modalities with regard to sampling interval and analyte detection range. Sensors that consume the sensed analyte, such as enzymatic and amperometric sensors, aggregate the sensed analyte over time, and therefore do not require a complete refreshing of the biofluid sample volume, and are not limited by instantaneous analyte concentrations in the biofluid. Accordingly, discussions of sampling rate and detection range herein will not apply to such sensors. On the other hand, sensors that equilibrate to the local analyte concentration, such as ion selective electrode and electrochemical aptamer-based sensors, will only produce new data at chronologically assured sampling intervals (i.e., when the sample volume is completely refreshed with new biofluid), and then will only register a measurement if the biofluid sample contains concentrations within the sensor's detection range.

For example, assume an electrochemical aptasensor for vasopressin is configured with a linear range of detection centered around vasopressin's normal concentration range in interstitial fluid, where the fluid is extracted by reverse iontophoresis. Unlike an amperometric sensor, the aptasensor does not consume the vasopressin, nor does the aptasensor aggregate detected vasopressin over time. Therefore, the vasopressin concentration in the biofluid must remain within the aptasensor's detection range for the sensor to detect vasopressin. Similarly, using a device with a slow biofluid refresh rate, such as GlucoWatch discussed above, the chronologically assured sampling interval for vasopressin would be in the multiple-hour range. Such sampling intervals would be entirely too slow for time-sensitive applications, like monitoring dehydration, or cortisol awakening response, which occurs within a 30 minute window after a person awakens, and requires multiple readings during that window.

The sensor modality also proves crucial for determining the biofluid sample generation rate required by the device. Returning to GlucoWatch, its glucose measurements in interstitial fluid depended on a sample generation rate that was largely determined and repeatable due to a controlled reverse iontophoresis current, and a 2 hour warm-up period to stabilize the extraction process. However, biofluid samples that include sweat are not so predictable, and generation rates can vary greatly depending on sweat rate. The introduction of sweat into GlucoWatch would confound its measurements, because not only do sweat and interstitial fluid have different glucose concentrations, but more importantly, the volume of sweat would increase the total biofluid generation rate, which would increase the total glucose captured in the gel, thereby giving a (false) glucose measurement.

### SUMMARY OF THE INVENTION

The devices described above, with a variable biofluid generation rate and that simply introduces a biofluid sample to sensors located within a large volume of gel or fluid are ineffective because: (1) for equilibrating sensors, the analyte concentration becomes diluted; and (2) for consuming sensors, the analyte concentration is dependent on sample generation rate.

Embodiments of the disclosed invention provide a device and method for accurate sensing with an analyte-consuming sensor in biofluids containing sweat. In an embodiment, a device for sensing a biofluid that is adapted to be placed on skin includes at least one analyte-consuming sensor for measuring at least a first analyte concentration of an analyte in a first biofluid sample having a first biofluid sample flow rate and at least one additional component that maintains analyte-consuming sensor measurements within 20% of the first concentration measurement when a biofluid sample flow rate is less than or equal to 2 times the first biofluid sample flow rate.

In another embodiment, a method for sensing a biofluid using a device adapted to be placed on skin includes measuring a first analyte concentration of an analyte in a first biofluid sample having a first biofluid sample flow rate using an analyte-consuming sensor, measuring the first biofluid sample flow rate, and maintaining a subsequent analyte concentration within 20% of the first analyte concentration when a subsequently measured biofluid flow rate is less than or equal to 2 times the first biofluid sample flow rate.

Additionally, according to an aspect of the present invention, the use of analyte-consuming sensors in a biofluid containing sweat must also accommodate the tremendous variability in pH and salinity presented by sweat. For example, pH variability between 5.0 and 7.0 (sweat can vary within this range) can cause the enzymatic activity of an enzymatic sensor to vary by as much as 50%. Salinity variations have similar, though less dramatic effects. Therefore, embodiments of the disclosed invention are directed to biofluid sensing devices using analyte-consuming sensors that track sample pH and salinity when sweat comprises part of the biofluid.

### BRIEF DESCRIPTION OF THE DRAWINGS

The objects and advantages of the disclosed invention will be further appreciated in light of the following detailed descriptions and drawings.

FIG. 1 is a cross-sectional view of a prior art wearable device for biosensing.

FIG. 2 is a cross-sectional view of a wearable device for biosensing according to an embodiment of the disclosed invention.

FIG. 3 is a cross-sectional view of a wearable device for biosensing according to an embodiment of the disclosed invention.

FIG. 4 is a cross-sectional view of a wearable device for biosensing according to an embodiment of the disclosed invention.

FIGS. 5-8 are cross-sectional views of portions of wearable devices for biosensing according to various embodiment of the disclosed invention showing multiple configurations of sensor(s) and catalyst region(s).

FIG. 9 shows graphs of voltage and analyte concentration versus time from an electrode with a high frequency sampling rate.

FIG. 10 shows graphs of voltage and analyte concentration versus time from an electrode with a low frequency sampling rate.

FIG. 11A is a schematic cross-sectional view of a device according to an embodiment of the disclosed invention.

FIG. 11B is a schematic cross-sectional view of the device of FIG. 11A after the fluid contacts the wicking component.

FIG. 11C is a schematic cross-sectional view of the device of FIG. 11A after the discrete sample of fluid has entered the wicking component and a graph of the current versus time over a sampling period.

FIG. 12A is a schematic cross-sectional view of a device according to an embodiment of the disclosed invention.

FIG. 12B is a schematic cross-sectional view of the device of FIG. 12A after fluid emerges from the opening of the chamber.

FIG. 12C is a schematic cross-sectional view of the device of FIG. 12A after the fluid contacts the wicking component.

FIG. 12D is a schematic cross-sectional view of the device of FIG. 12A after the discrete sample of fluid has entered the wicking component.

FIG. 13 is an enlarged cross-sectional view of the encircled portion of FIG. 12B.

### DEFINITIONS

As used herein, “interstitial fluid” or “tissue fluid” is a solution that bathes and surrounds tissue cells. The interstitial fluid is found in the interstices—the spaces between cells (also known as the tissue spaces). Embodiments of the disclosed invention described herein focus on interstitial fluid found in the skin and, particularly, interstitial fluid found in the dermis. In some cases where interstitial fluid is emerging from sweat ducts, the interstitial fluid contains some sweat as well, or alternately, sweat may contain some interstitial fluid. As used herein, “mainly interstitial fluid” means fluid that contains by volume less than 50% sweat (i.e., is primarily interstitial fluid). As used herein, “mainly sweat” means fluid that contains by volume 50% or greater of sweat (i.e., may contain some interstitial fluid, but has equal or greater amount of sweat than interstitial fluid). The percentages of each fluid can be quantified by several methods, such as measuring analyte dilutions in sweat (e.g., some analytes are dilute in sweat but not in interstitial fluid), or by measuring and comparing sample generation rates; their respective contributions to the total fluid volume quantified (e.g., compare sample generation rates with or without application of reverse iontophoresis or compare sample generation rates with or without natural or chemically-induced sweat stimulation).

As used herein, “biofluid” is a fluid that is comprised mainly of interstitial fluid or sweat as it emerges from the skin. For example, a fluid that is 45% interstitial fluid, 45% sweat, and 10% blood is a biofluid as used herein. For example, a fluid that is 20% interstitial fluid, 20% sweat, and 60% blood is not a biofluid as used herein. For example, a fluid that is 100% sweat or 100% interstitial fluid is a biofluid. A biofluid may be diluted with water or other solvents inside a device because the term biofluid refers to the state of the fluid as it emerges from the skin. Generally, as compared to blood, sweat is highly dilute of large sized analytes (e.g., greater than 1000× for proteins, etc.). To a lesser extent, as compared to blood, interstitial fluid is dilute for some larger sized analytes (e.g., 10 to 100X or more or less depending on the specific analyte, current density, etc.).

As used herein, “interstitial fluid sampling rate” or “sweat sampling rate” or simply “sampling rate” is the effective rate at which a new biofluid sample, originating from the pre-existing pathways, reaches a sensor that measures a property of the fluid or its solutes. Sampling rate is the rate at which new biofluid is refreshed at the one or more sensors and therefore old biofluid is removed as new fluid arrives. In an embodiment, this can be estimated based on volume, flow-rate, and time calculations, although it is recognized that some biofluid or solute mixing can occur. Sampling rate directly determines or is a contributing factor in determining the chronological assurance. Times and rates are inversely proportional (rates having at least partial units of 1/seconds), therefore a short or small time required to refill sample volume can also be said to have a fast or large sampling rate. The inverse of sampling rate (1/s) could also be interpreted as a “sampling interval” (s). Sampling rates or intervals are not necessarily regular, discrete, periodic, discontinuous, or subject to other limitations. Like chronological assurance, sampling rate may also include a determination of the effect of potential contamination with previously generated biofluid, previously generated solutes (analytes), other fluid, or other contamination sources for the measurement(s). Sampling rate can also be in part determined from solute generation, transport, advective transport of fluid, diffusion transport of solutes, or other factors that will impact the rate at which new sample will reach a sensor and/or is altered by older sample or solutes or other contamination sources. During reverse iontophoretic extraction of fluid samples and analytes, some analytes that have a net charge could move faster or slower, with or against, the advective flow of fluid sample. In the event that the analytes are moving faster or slower than the advective flow, the sampling rate is still determined by the advective flow of interstitial fluid and the replenishment of new fluid sample across the sensor as the old sample is replaced. If an embodiment of the disclosed invention does not include a net flow of sample fluid across a sensor, and does include transport of a solute (analyte) to the sensor, then the term sampling rate may be replaced with the term “analyte sampling rate”. As will be described in greater detail below, sampling rate may be interpreted with respect to equilibrating sensors as part of the process of sensing the analyte, because such sensors depend on a flow of fresh analyte to the sensors and removal of old analyte away from the sensors.

As used herein, “sweat stimulation” is the direct or indirect causing of sweat generation by any external stimulus. One example of sweat stimulation is the administration of a sweat stimulating chemical, such as pilocarpine or carbachol, from a sweat stimulating component. Going for a jog, which stimulates sweat, is sweat stimulation, but would not be considered as a sweat stimulating component. Sweat stimulation can include sudo-motor axon reflex sweating, passively diffusing a chemical into skin to stimulate sweat, or any other suitable method for sweat stimulation. As further examples, sweat stimulation can be achieved by simple thermal stimulation, by orally administering a drug, by intradermal injection of drugs such as methylcholine, carbachol, or pilocarpine, and by dermal introduction of such drugs using iontophoresis.

As used herein, “measured” can imply an exact or precise quantitative measurement and can include broader meanings such as, for example, measuring a relative amount of change of something. Measured can also imply a binary measurement, such as ‘yes’ or ‘no’ type qualitative measurements.

As used herein, “sample volume” is the fluidic volume in a space that can be defined multiple ways. Sample volume may be the volume that exists between a sensor and the point of generation of biofluid sample. Sample volume can include the volume that can be occupied by sample fluid between: the sampling site on the skin and a sensor, where the sensor has no intervening layers, materials, or components between it and the skin; or the sampling site on the skin and a sensor, where there are one or more layers, materials, or components between the sensor and the skin.

As used herein, “microfluidic components” are channels or other geometries formed in or by polymers, textiles, paper, or other components known in the art to transport fluid in a deterministic manner.

As used herein, “advective transport” is a transport mechanism of a substance or conserved property by a fluid due to the fluid's bulk motion.

As used herein, “diffusion” is the net movement of a substance from a region of high concentration to a region of low concentration. This is also referred to as the movement of a substance down a concentration gradient.

As used herein, the term “analyte-specific sensor” is a sensor specific to an analyte and performs specific chemical recognition of the analyte's presence or concentration (e.g., ion-selective electrodes, enzymatic sensors, electrochemical aptamer based sensors, etc.). For example, sensors that sense impedance or conductance of a fluid, such as biofluid, are excluded from the definition of “analyte-specific sensor” because sensing impedance or conductance merges measurements of all ions in biofluid (i.e., the sensor is not chemically selective; it provides an indirect measurement). Sensors could also be optical, mechanical, or use other physical/chemical methods that are specific to a single analyte. Further, multiple sensors can each be specific to one of multiple analytes.

As used herein, the term “analyte-consuming sensor” is an analyte-specific sensor that decreases the total amount of analyte present (e.g., enzymatic or amperometric sensing).

As used herein, the term “equilibrating sensor” is an analyte-specific sensor that responds by equilibrating to the local concentration of the analyte (e.g., ionselective or electrochemical aptamer-based sensors) and that does not decrease the total amount of the analyte present. An aptasensor may bind an analyte, but the analyte is not consumed (i.e., once the analyte binds, the same site will not bind further analyte, and furthermore, the analyte can be released back into solution as well). The definition and calculations for sampling rate and sampling interval described herein apply to cases using equilibrating sensors.

As used herein, “concentration regulating component” is any component that regulates concentration of analyte around an analyte-consuming sensor. As such, errors due to sample generation rate or sample flow rate are mitigated, and flow rates may vary by at least 2× without a 20% change in sensor readings.

As used herein, “diffusion limiting material” is any material or component between a sensor and biofluid that limits the diffusion of an analyte to an analyte-consuming sensor compared to the case of such a sensor being directly exposed to the biofluid. As such, errors due to sample generation rate or sample flow rate are mitigated, and flow rates may vary by at least 2× without a 20% change in sensor readings. A diffusion limiting material may be a concentration regulating component.

### DETAILED DESCRIPTION OF THE INVENTION

Embodiments of the disclosed invention apply at least to any type of sensing device that measures at least one analyte in a biofluid comprising sweat or interstitial fluid. Further, embodiments of the disclosed invention apply to sensing devices that measure at chronologically assured sampling intervals or sampling rates. Further, embodiments of the disclosed invention apply to sensing devices that can take on forms including patches, bands, straps, portions of clothing, wearables, or any suitable mechanism that reliably brings sampling and sensing technology into intimate proximity with a biofluid sample as it is transported to the skin surface. Some embodiments of the disclosed invention may utilize adhesives to hold the device near the skin, while devices could also utilize other mechanisms that hold the device secure against the skin, such as a strap or embedding in a helmet. Certain embodiments of the disclosed invention describe sensors as simple individual elements. It is understood that many sensors require two or more electrodes, reference electrodes, or additional supporting technology or features which, for the sake of brevity, may not be captured in the description herein. Sensors are preferably electrical in nature, but may also include optical, chemical, mechanical, or other known biosensing mechanisms. Sensors may be analyte-consuming sensors, such as enzymatic sensors. Sensors can be in duplicate, triplicate, or more, to provide improved data and readings. Certain embodiments of the disclosed invention show sub-components of what would be sensing devices with more sub-components needed for use of the device in various applications, which are obvious (such as a battery), and for purposes of brevity and of greater focus on inventive aspects, such components are not explicitly shown in the diagrams or described in the embodiments of the disclosed invention.

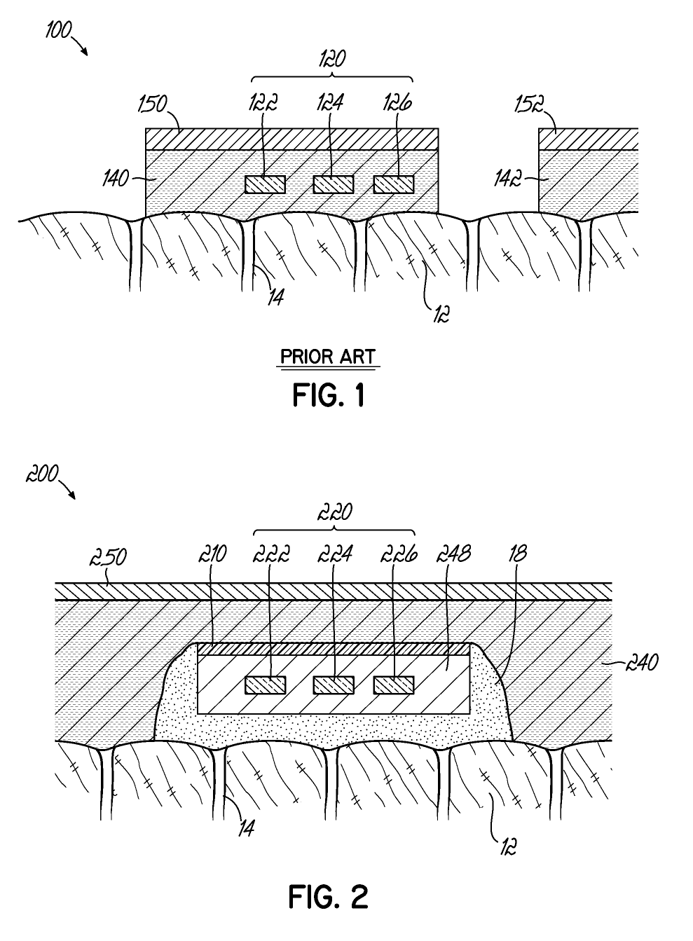

With reference to FIG. 1, a portion of a prior art device 100 is shown and positioned on the skin 12. The device 100 includes a gel 140, such as agar, that may optionally contain a sweat stimulant, such as pilocarpine, and an optional electrode 150 such as silver or carbon. The electrode 150 can be used for iontophoretic delivery of the sweat stimulant to the skin 12 to cause sweating and/or for reverse iontophoresis generation of a flow of biofluid to the skin surface. The device 100 further includes at least one analyte-consuming sensor 120 having a reference electrode 122, counter electrode 124, and working electrode 126. The working electrode 126 is coated with a material (not shown), such as an enzyme, which consumes the analyte (e.g., glucose oxidase to consume glucose). The working electrode 126 may be configured with any other suitable coating, such as those for detecting lactate, ethanol, etc. The device 100 further includes a counter electrode 152 to electrode 150 and a gel 142 for electrical contact with the skin 12. If a biofluid sample is generated by reverse iontophoresis only, then doubling the reverse iontophoresis current would have a predictable increase in the interstitial fluid generation rate. Therefore, an increase in concentration of the analyte in the gel 140 could be measured by sensor 120, and the device 100 could make a meaningful determination of analyte concentration in the biofluid. However, if sweat is present in the biofluid, the sample generation rate may vary significantly and would no longer be predictable by the device 100. For example, any number of daily factors, such as entering a hot room, walking, eating, becoming nervous, etc., could cause the sweat rate to increase for unpredictable durations or magnitudes, even where a steady baseline of sweat is generated by artificial means. Such unpredictable changes in biofluid sample generation rate may not be measureable by the device 100, rendering accurate analyte measurements impossible.

With reference to FIG. 2, in an embodiment of the disclosed invention, a portion of a device 200 includes a gel 240 and an analyte-consuming sensor 220, which contains a reference electrode 222, counter electrode 224, and working electrode 226. The working electrode 226 is specific to an analyte and consumes the analyte as it measures the concentration (i.e., it is an analyte-consuming electrode). The biofluid 18 may be stimulated or naturally flowing. As biofluid 18 enters the gel 240, the analytes in the biofluid 18 will be diluted due to the relatively large volume of the gel 240. Thus, if the sensor 220 received biofluid 18 from the gel 240, any measurements taken may not be accurate. Accordingly, the device 200 further includes a diffusion limiting material 248 and a sweat impermeable substrate 210, which reduces the contact area between the diffusion limiting material 248 and the gel 240. The diffusion limiting material 248 surrounds the sensor 220 so that the sensor 220 is not in contact with the gel 240. The diffusion limiting material 248 is configured such that it is rigid enough to support the substrate 210 and can accommodate a volume of biofluid 18, such as sweat, between the substrate 210 and the skin 12. As a result, the diffusion limiting material 248 is exposed directly to an advective flow of the biofluid 18 as it emerges from the skin 12. Thus, the sensor 220 receives the analyte from the biofluid 18 as the analyte moves through the diffusion limiting material 248, rather than receiving biofluid 18 from the gel 240. The biofluid 18 itself does not pass through the diffusion limiting material 248. The rate at which analyte from the biofluid 18 reaches the sensor 220 is limited by the rate at which the analyte may diffuse through the diffusion limiting material 248. Consequently, a change in the flow rate of the biofluid 18 from the skin 12 will not affect the local balance between the analyte flowing from the skin 12 and the consumption of the analyte by the sensor 220. As a result, the sample generation rates or sample flow rates may vary by at least 2× without causing more than a 20% change in sensor readings. Such percentages can be validated by calibrating the device prior to use with a calibration solution (e.g., constant flow rate and changing analyte concentration, and calibrating with changing flow rate and constant analyte concentration). Exemplary diffusion limiting materials include a higher weight percentage agar gel (e.g., 20% or more), a nano-porous filtration membrane, a dialysis membrane, a track-etch membrane, or other suitable materials. Because these diffusion-limiting materials at minimum limit diffusion of one or more analytes to the sensor 220, they can be applied to the sensor 220, for example, as a conformal coating or as a loose film (i.e., an unattached film). In an embodiment, a conformal coating could be a polymer, such as PVC, that is applied by solution coating and rapidly dried by heat or vacuum to create a nano-porous coating. Further, a conformal coating could be a slurry or nano-bead composite with a weak binder or a self-binding effect (e.g., flocculation, sedimentation). In an embodiment, a loose film could be a track-etch membrane held with pressure against the sensor (e.g., pressure caused by a wicking material or channel pushed against the film and sensor). A loose film may be appropriate, for example, if there are relatively long pathways necessary to move analytes through the loose edges of the film, which therefore also creates a diffusion-limiting effect. Diffusion limiting materials, in some cases, will allow diffusion and removal of byproducts of the sensor 220, especially byproducts of enzymatic sensors that breakdown or convert the analyte into other chemical constituents that may comprise a byproduct.

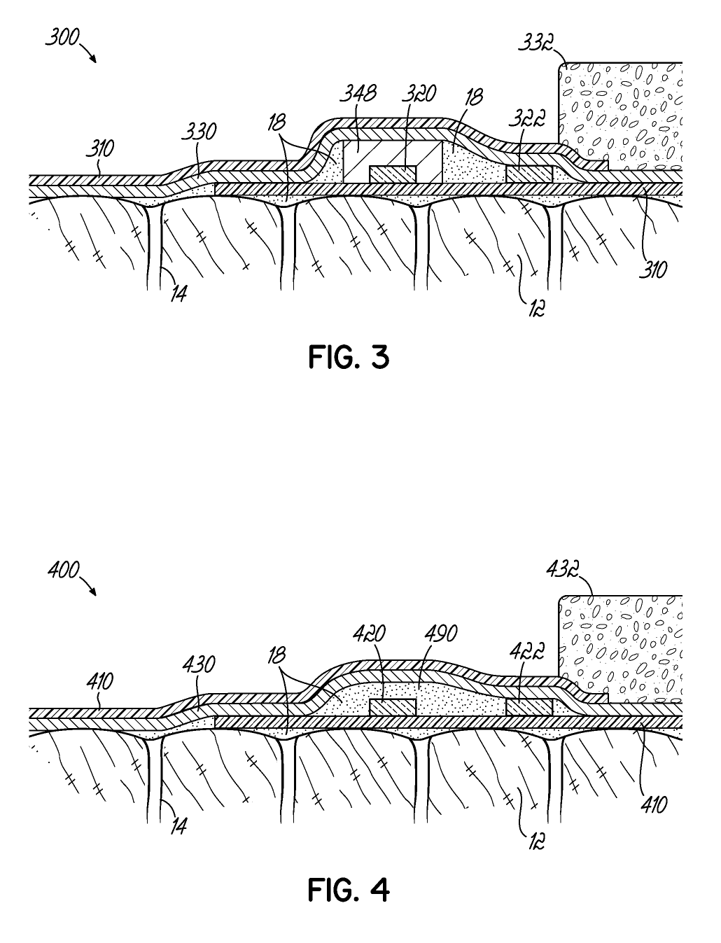

With reference to FIG. 3, in an embodiment of the disclosed invention, a portion of a device 300 includes a diffusion limiting material 348 and a wicking pump 332, such as a hydrogel, to remove old or excess sweat. The device 300 further includes a wicking component 330, such as paper, Rayon textile, membrane, or microfluidic channel, to bring biofluid 18 into the device 300 and into contact with the sensors 320, 322 and eventually into the wicking pump 332. The sensor 322 could be a secondary sensor used to further improve accuracy of measurements taken by the primary analyte-consuming sensor 320. For example, the secondary sensor 322 could measure one or more of the following: sweat onset; sample generation rate; skin impedance; sweat conductivity; galvanic skin response (GSR); biofluid sample pH; device temperature; micro-thermal flow rate; salinity; or an ion or other analyte concentration that is proportional to sweat generation rate, e.g., Cl— or lactate. For other embodiments, rather than, or in addition to, the secondary sensor 322 being configured to measure the pH of the biofluid 18, the analyte-consuming sensor 320 may be configured to indirectly measure the pH by measuring the redox of protons. Alternately, the analyte-consuming sensor 320 could be configured to measure a redox active metabolite, such as FADH₂/FAD⁺, NADH/NAD⁺, NADPH/NADP⁺, or other biological or chemical redox molecules. In another embodiment, the device 300 may also detect the metabolite indirectly by, for example, measuring an electrical signal. For example, glucose oxidase generates peroxide, which reacts with Prussian blue (C₁₈Fe₇N₁₈) to produce a measureable electrical signal. All such configurations are contemplated within the scope of the present disclosure.

In an embodiment of the disclosed invention, one or more of these secondary measurements may be used to convert the output of the analyte-consuming sensor into an analyte concentration. For example, a device measuring sweat glucose with an analyte-consuming sensor uses a secondary sensor to measure a biofluid pH. Using an algorithm, formula, look-up table, or other suitable means, the device would identify an expected enzymatic activity level for the measured pH, and then correct the glucose measurement accordingly. Similarly, the secondary sensor may be a sweat conductivity sensor, Na+ISE, or other sensor for measuring biofluid salinity and biofluid sample flow rate. Salinity measurements are used in a similar manner to the pH measurements to correct for enzymatic activity level changes. Biofluid sample flow rate measurements may further correct the glucose sensor by providing the device with information on biofluid volume or flow rate variability. For example, the device may be configured to measure glucose concentrations within a sample flow rate range of 2×. With a measurement of sample flow rate, an algorithm or other suitable method may be used to correct the measured glucose concentration for the volume of sample that has actually passed across the glucose sensor. For example, an algorithm could use the measured sample flow rate to provide an estimated sample volume from time 0 to time t. Using the output from the analyte-consuming sensor, the algorithm could also determine the moles of glucose measured from time 0 to time t. The device would then have a total glucose concentration value for the period time 0 to time t. In an embodiment where the secondary sensor 322 is a GSR sensor, the salinity and sample flow rate measurements may be further improved by determining sweat onset and identifying periods of increasing or decreasing sweat rate.

With further reference to FIG. 3, in an embodiment, the secondary sensor 322 could also be specific to the same analyte sensed by the primary sensor 320. For example, both sensors 322, 320 could be specific to glucose. The diffusion limiting material 348 creates a diffusion-limited environment for primary sensor 320, which in most cases, improves sensor accuracy. However, the analyte diffusion rate through the diffusion limiting material 348 will increase with increasing analyte concentrations in the biofluid sample. Therefore, during periods of increased analyte concentration, increased analyte consumption by the primary sensor 320 can locally lower analyte concentrations inside the diffusion limiting material 348, causing the primary sensor 320 to (inaccurately) measure a stable analyte concentration during a period when the concentration is actually increasing. With the secondary sensor 322 configured to measure the same analyte, it may measure any remaining analyte that diffuses through the diffusion limiting material 348. The measurements of the two sensors 320, 322 may then be integrated to provide an accurate total for analyte molecules received by the device 300.

With reference to FIG. 4, in an embodiment of the disclosed invention, a portion of a device 400 includes a sweat impermeable substrate 410, a wicking component 430, and a volume 490 through which an advective flow of the biofluid 18 may move. As biofluid 18 emerges from the skin 12, the biofluid 18 moves through the wicking component 430 towards a wicking pump 432. The volume 490 is adjacent to the wicking component 430 so that, as biofluid 18 moves through the wicking component 430, biofluid 18 enters the volume 490. Sensors 420, 422 of the device 400 are carried on the sweat impermeable substrate 410 and are positioned within the volume 490. As a result, if the flow rate of new sample is high enough (e.g., 50 nL/min), the sensor 420 is small enough (e.g., 50×50 μm²), and the volume 490 around the sensor 420 is large enough (e.g., 200 nL), then the analyte-consuming sensor 420 will not be able to consume enough analyte to significantly alter the analyte concentration in the volume 490 (e.g., a change of not more than 20% change in sensor signal over flow rate changes of 2×, such as from 1 to 2 nL/min/gland). Thus, the volume 490 allows the analyte-consuming sensor 420 to measure analyte concentrations accurately over a range of biofluid flow rates. Further, the secondary sensor 422 is in contact with the wicking component 430. Accordingly, the secondary sensor 422 is in contact with the biofluid 18 that moves through the wicking component 430 unlike the analyte-consuming sensor 420. The secondary sensor 422 could be a sensor that measures one or more of sweat onset, sample generation rate, pH, device temperature, or salinity to further improve accuracy of measurements taken by analyte-consuming sensor 420. The volume 490 could be created by the use of concentration regulating component, which may be defined by a gel, microfluidic geometry, or other suitable method. The volume 490 could also be simply open space. Other exemplary concentration regulating components include beads packed with aptamers that bind and release the analyte, which could serve a purpose similar to that shown for the volume 490. Alternate concentration regulating components could also be the diffusion limiting materials described herein. However, using a concentration regulating component as described will result in slower sampling intervals and therefore longer times between chronologically assured analyte measurements. To ensure chronological assurance, sample volumes and sample flow rates can be determined or measured (e.g., thermal flow meters, not shown) to provide the rate at which fresh samples of biofluid reach the sensors.

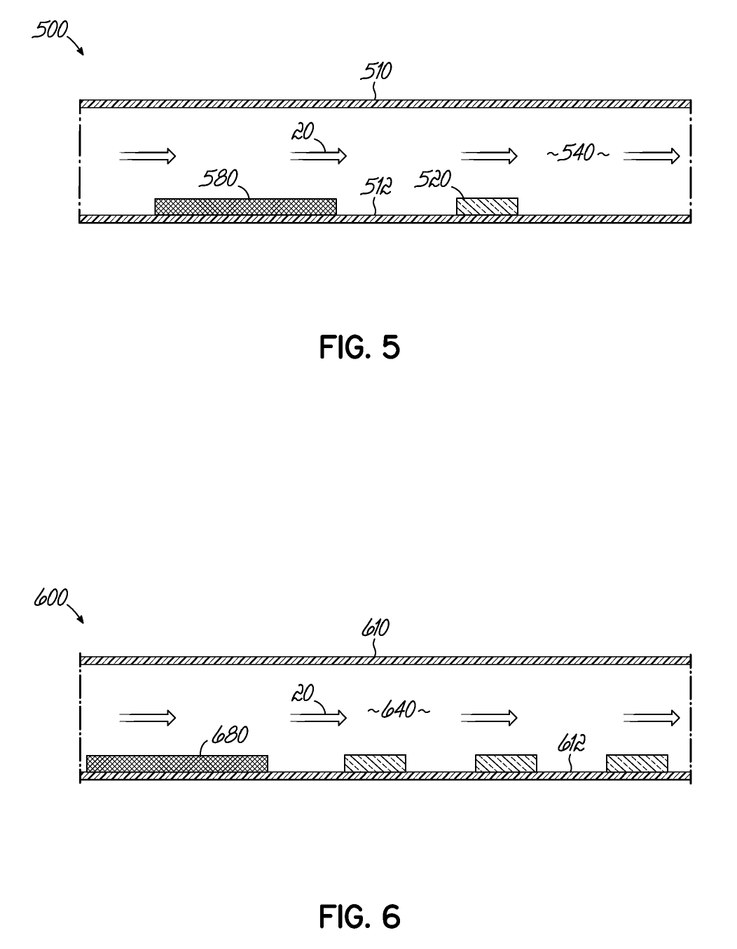

Another embodiment of the disclosed invention includes a microfluidic channel or wicking material coinciding with, or arranged in parallel to, the flow of biofluid 18 through the device. With reference to FIG. 5, in an embodiment of the disclosed invention, a device includes a sensing channel 500 of known volume 540 that is bound by a top fluid impermeable substrate 510 and a bottom fluid impermeable substrate 512. For example, with further reference to FIG. 4, the sensing channel 500 may be arranged in parallel with the wicking component 430 or, alternatively, may be the same component as the wicking component 430. An analyte-consuming sensor 520 is positioned in the sensing channel 500. The direction of the flow of the biofluid 18 through the sensing channel 500 and across the analyte-consuming sensor 520 is depicted by an arrow 20.

For some analytes, it may be difficult to develop a suitable analyte-consuming sensor for use in sweat sensing devices. In such cases, it may prove more convenient to include a sensor that can detect a transformed version of the analyte, for example a product of the analyte after interaction with a catalyst. Therefore, in some embodiments, the analyte-consuming sensor includes a catalyst. With reference again to FIG. 5, upstream of the sensor 520 relative to the flow of biofluid 18 is a catalyst region 580 that is functionalized with a catalyst that is selected to facilitate measurement of the target analyte for the sensor 520. The catalyst region 580 is in fluid communication with the analyte-consuming sensor 520. The catalyst can be an enzyme, for example, a dehydrogenase, an oxidase, or a deglycosylated enzyme. In other cases, the catalyst may be an RNAzyme, a DNAzyme, or a polymeric matrix. The catalyst region 580 is sized (i.e., contains sufficient quantity of enzyme) so that the total analyte concentration present in the sample of biofluid 18 as it flows across the catalyst region 580 is converted by the catalyst into a species, which may be referred to as a mediator, that can be detected by the sensor 520. The size and/or catalyst quantity of the catalyst region 580 for achieving this can be determined by considering enzyme kinetics, diffusion kinetics, biofluid flow rates, etc., according to techniques known in the art. Enzyme functionalization within the catalyst region 580 can be by direct immobilization of the enzyme on a surface exposed to the biofluid 18 or by immobilization of the enzyme within a suitable matrix. Suitable materials for the matrix that immobilizes an enzyme include, without limitation, coated silica nanoparticles, cellulose acetate films, gels, and conducting polymers. Examples of suitable gels include polyacrylamide, agarose, and chitosan. Examples of suitable conducting polymers include polypyrrole; poly(N-(4-(3-thienyl methylene)-oxycarbonyl phenyl) maleimide-co-pyrrole); a polyaniline film; charged polysaccharides (carboxymethylpullulan); chitosan; and poly(5-hydroxy-1,4-naphthoquinone-co-5-hydroxy-3-acetic acid-1,4-naphthoquinone).

Some embodiments of the disclosed invention may benefit from having multiple analyte-consuming sensors in a sensing channel With reference to FIG. 6, where like numbers reference like components as depicted in FIG. 5, the sensing channel 600, which is defined at least in part by the sweat impermeable materials 610, 612, includes a plurality of analyte-consuming sensors 620, 622, 624. The plurality of analyte-consuming sensors 620, 622, 624 are arranged along the sensing channel 600 so that, when biofluid 18 flows into the device, it will be carried across the sensors 620, 622, 624 sequentially. By comparing the signals produced by the sensors 620, 622, 624 as the sample flows through the channel 600, the device may determine a sample flow rate. For example, the channel 600 may be a microfluidic wick within a device, and the analyte consuming sensors 620, 622, 624 may be configured to measure vasopressin. As a sample of the biofluid 18 flows through the sensing channel 600 and across the sensors, the first sensor 620 measures a first concentration of vasopressin, the second sensor 622 measures a second concentration of vasopressin, and the third sensor 624 measures a third concentration of vasopressin. The device would compare the three concentrations and the time at which each concentration was measured to determine a sample flow rate. Further, having multiple analyte-consuming sensors allows the device to continue accurately measuring analyte concentrations, even if one or more of the sensors become saturated with analyte. For example, if the first sensor 620 became saturated with glucose, unconsumed glucose would flow downstream to the second sensor 622 or to the third sensor 624, if the second sensor 622 were also saturated. The device can then add the concentrations measured by each of the sensors 620, 622, 624 to determine a total amount of glucose flowing into the sensing channel 600.

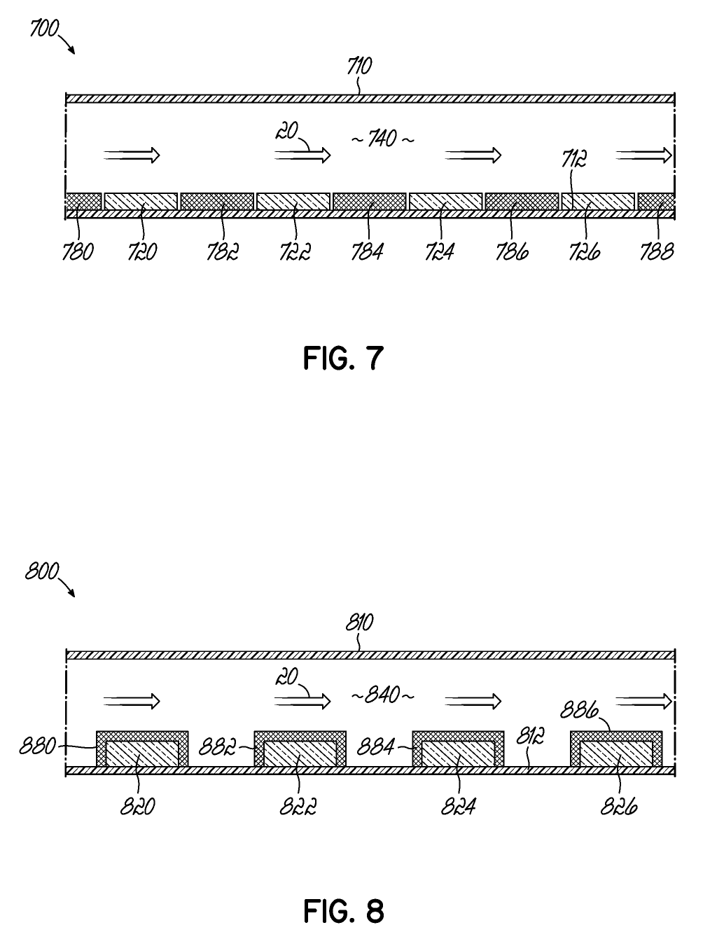

With reference to FIGS. 6-8, the number and arrangement of catalyst region(s) in various embodiments may vary. For example, as shown in FIG. 6, the single catalyst region 680 may be located upstream of all of the sensors 620, 622, 624. As shown in FIG. 7, a plurality of catalyst regions 780, 782, 784, 786, 788 are located upstream of a plurality of analyte-consuming sensors 720, 722, 724, 726. More specifically, the catalyst regions 780, 782, 784, 786, 788 alternate with the analyte-consuming sensors 720, 722, 724, 726. Thus, within each catalyst region and analyte-consuming sensor pair (e.g., 780, 720), the catalyst region is upstream of the analyte-consuming sensor. The catalyst region/sensor arrangement may take other suitable forms, so long as the target analyte is effectively converted into a detectable species, as needed by the application. For example, with reference to FIG. 8, each sensor 820, 822, 824, 826 is covered by, or coated with, a catalyst region 880, 882, 884, 886 (e.g., an enzyme coating or immobilization layer). In this configuration, each sensor 820, 822, 824, 826 is co-located with one of the catalyst regions 880, 882, 884, 886 so that analyte flowing to each of the sensors 820, 822, 824, 826 will first interact with a catalyst region 880, 882, 884, 886.

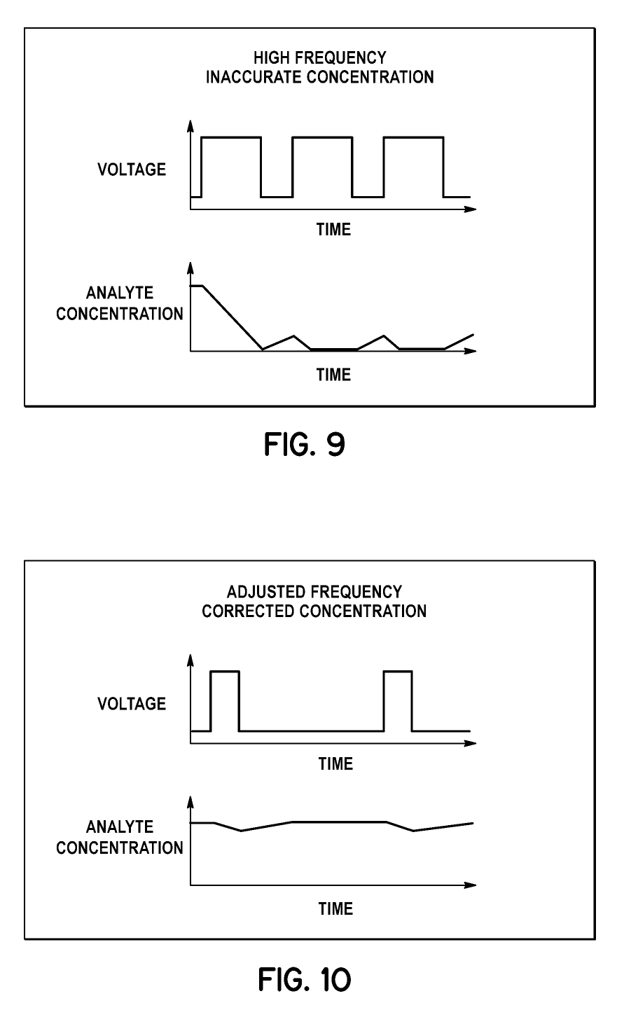

Embodiments with an analyte-consuming sensor may include methods and devices for measuring an analyte in a continuous system irrespective of sample flow rate or volume size by digitized or discrete sampling. In various embodiments, the discrete sampling includes (1) electrical pulses and/or (2) a discrete volume dosing system. Discrete sampling according to embodiments of the disclosed invention allows for accurate measurement of an analyte concentration when there is a low flow rate and/or volume size. For example, FIG. 9 shows a high frequency of pulses that provides an inaccurate concentration measurement at a slow flow rate. FIG. 10 shows an adjusted frequency of pulses that provides a more accurate or corrected concentration at the same flow rate. The pulse duration (t_{d}) depends on the fluid volume, requiring a shorter pulse for smaller volumes or flow rates of the biofluid to a sensor. As an example, the pulse duration may be less than 10 seconds when the volume is less than 20 μL/min and the supply rate is 1 μL/min (for a voltage of 0.6V). The pulse time will vary as the flow rate increases or decreases.

In an embodiment, short electrical pulses shorten the amount of the mediator or catalyzed product that is reduced/oxidized by the catalyst. The frequency of the pulses may be adjusted based on the flow rate of the target analyte. Short sampling ensures that not all of the mediator is consumed, and slower flow rates of the target analyte support the consumption rate of the mediator. If the flow rate is known using a flow meter, the frequency or duration of electrical pulses can be adjusted accordingly in software if the flow rate changes

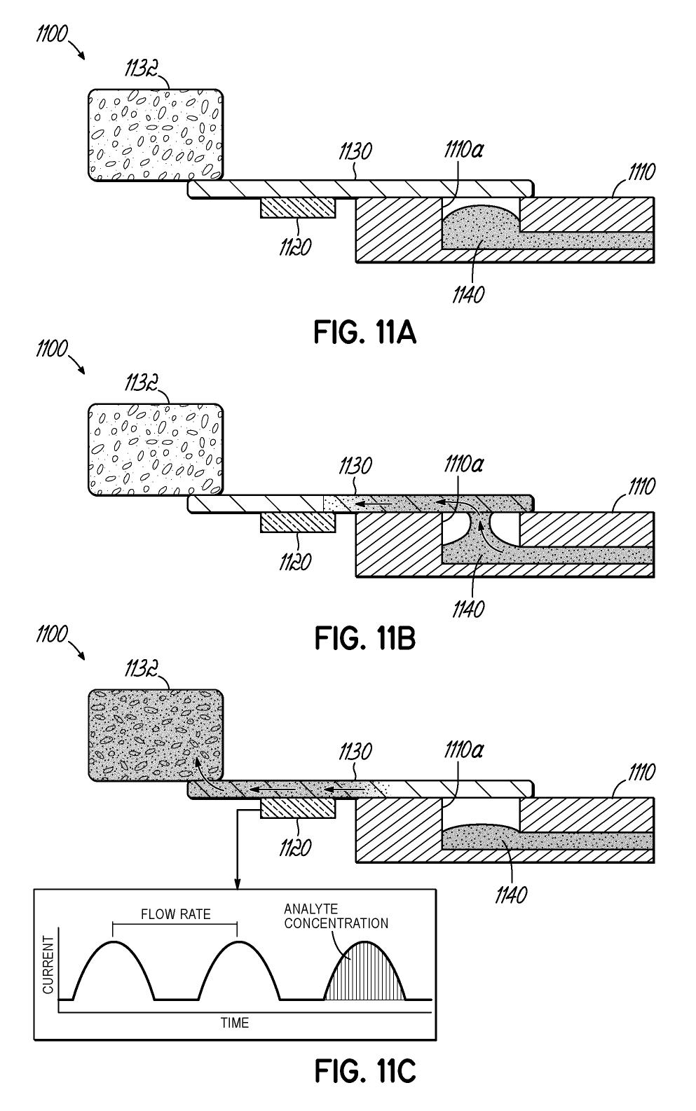

With reference to FIGS. 11A-11C, in an embodiment including an analyte-consuming sensor, a discrete volume dosing system comprises a biofluid sensing device 1010, which is a closed or sealed system in which discrete, quantized samples of fluid are delivered and analyzed independent of flow rate. The device 1100 includes a fluid-impermeable chamber 1110 that includes an opening 1110a. The chamber 1110 may be made of, for example, acrylic. The chamber 1110 defines a fluid channel 1140, which may be coated with a hydrophobic material (e.g., Teflon or silica-gel). The channel 1140 is designed to receive a continuous, pressure-driven flow of sample fluid. The sample fluid travels through the channel 1140 towards the opening 1110a. The device 1100 further includes a wicking component 1130 (e.g., Rayon or polyester fibers) at least a portion of which is adjacent to the opening 1110a of the chamber 1110. The wicking component 1130 transports fluid from the channel 1140 and delivers the discrete samples to an analyte sensor 1120. Although not shown, a diffusion-limiting material such as one described above may act as a barrier between the wicking component 1130 and the sensor 1120. A pump 1132 is in fluidic contact with the wicking component 1130 and aids in drawing the sample fluid through the wicking component 1130 and across the analyte sensor 1120 (or the diffusion-limiting material when present). Suitable materials for the pump 1132 include sodium polyacrylate or a wicking material.

As shown in FIG. 11A, because of the hydrophobic coating, a convex meniscus forms. As more fluid enters the channel 1140, the convex meniscus moves towards and eventually contacts the wicking component 1130. Referring to FIGS. 11B and 11C, when the meniscus contacts the wicking component 1130, spontaneous capillary flow occurs and a droplet of the sample fluid enters the wicking component 1130. As the droplet of the sample fluid travels through the wicking component 1130, the fluid in the channel 1140 loses contact with the wicking component 1130. Thus, the separate sample droplets are formed from each other due at least in part to capillary forces. The meniscus contacts the wicking component 1130 only when a specific volume is reached. The volume of the droplet depends on several factors, but primarily the height between the chamber or the opening and the wick (see FIG. 13). As more fluid enters the channel 1140, the process is repeated. The device is flow rate independent (i.e., flow rate can vary or even be erratic), and the droplet only enters and moves through the wicking component 1130 and past the analyte sensor 1120 when the predetermined volume is reached. Thus, the wicking component 1130 transports a series of discrete, quantized samples of the fluid across the analyte sensor 1120 (or the diffusion-limiting material when present). There is a reaction area adjacent the analyte sensor 1120 in which all or a part of the analyte is consumed or reacted. As each discrete sample contacts and passes over the analyte sensor 1120, the measured current will increase rapidly and then decrease as the analyte is consumed by the sensor 1120 or as it flows away from the sensor 1120 (FIG. 11C). In an embodiment, the concentration of the analyte is measured by the area under the current v. time curve. In addition, the flow rate of the sample is calculated by measuring the periodicity of each sample.

Low (or changing) flow rates (e.g., less than 20 μL/min) and low volume (e.g., less than 20 μL) fluids are typically difficult or impossible to accurately measure analyte concentration since the analytes are consumed rapidly and skew the results. For example, as flow rate increases in small volumes, it appears that the analyte concentration increases since more analyte is consumed. In an aspect of the disclosed invention, measurements are improved by combining analyte consumption rates and volumetric dispensing of fluid samples to ensure that the concentration of the analytes is accurately measured (i.e., the area under the curve) for continuous flow systems. In addition to concentration, the flow rate of the sample is also directly sampled by measuring the periodicity of each sample in each rise in current for each time a packet of fluid is received.

In an aspect of the disclosed invention, the fluid supply to the analyte sensor 1120 may be active or passive. For example, the device 1100 includes passive, spontaneous capillary flow to provide samples of the fluid to the analyte sensor 1120. In an embodiment, a discrete volume dosing system with active fluid supply may include a sensor that detects when a sufficient amount of fluid is present and a pump that dispenses a sample of the fluid accordingly.

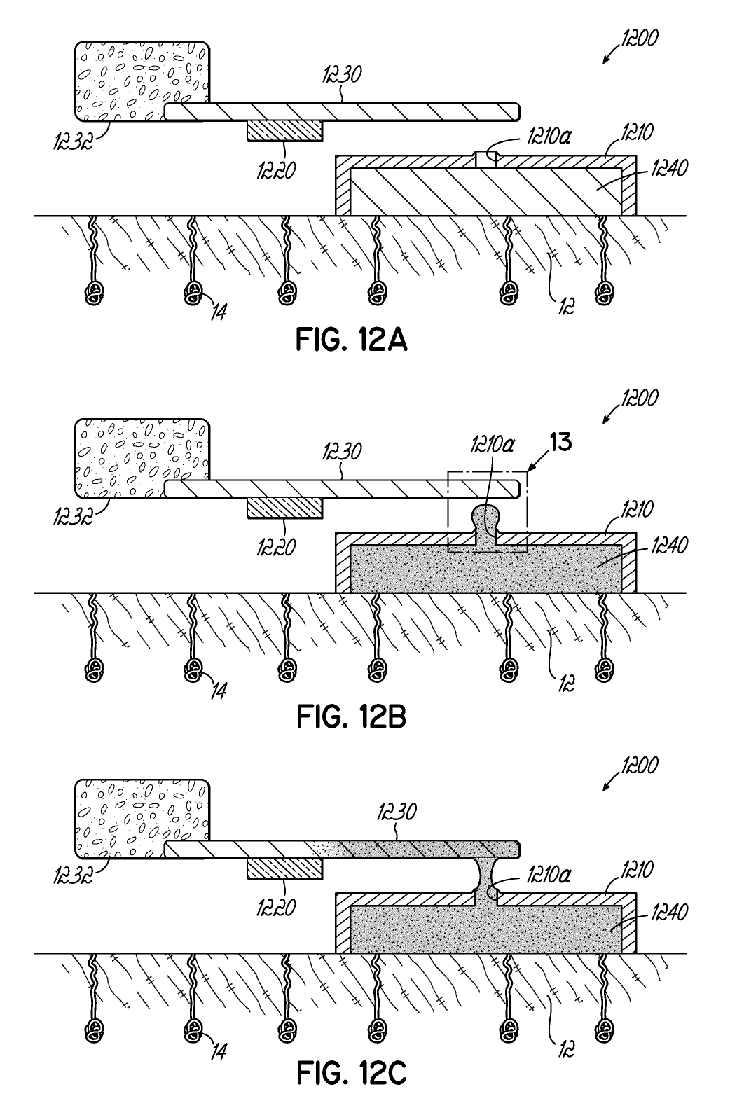

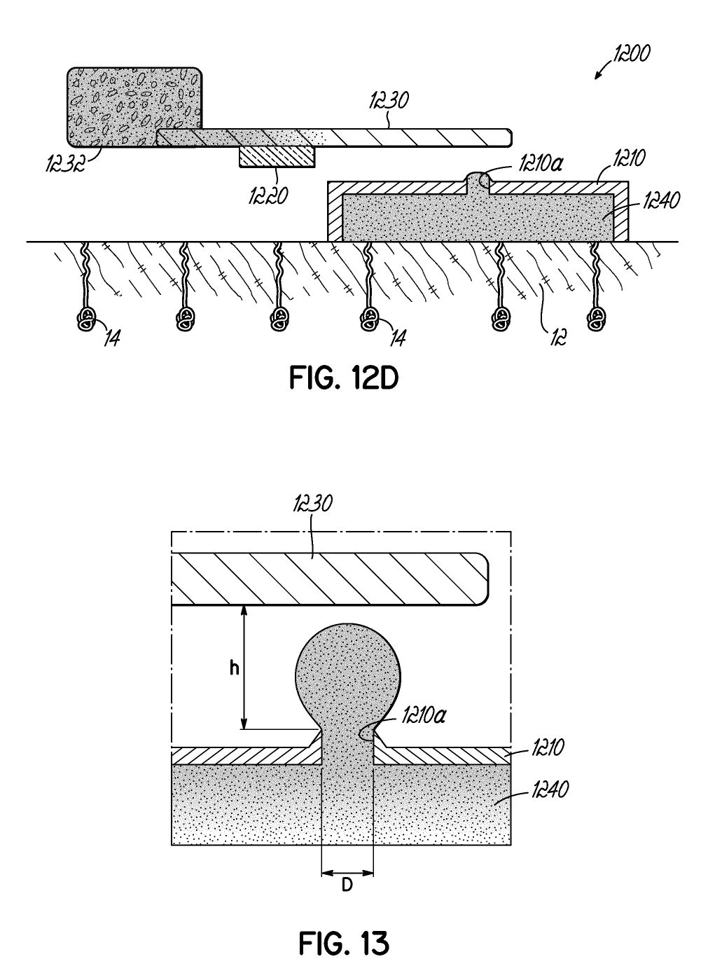

With reference to FIGS. 12A-12D, in another embodiment including an analyte-consuming sensor, a discrete volume dosing system with active fluid supply is shown. The discrete volume dosing system includes a device 1200 that may be sealed to the skin 12 (e.g., through tape or other adhering techniques). The device 1200 includes a fluid-impermeable barrier 1210 that includes an opening 1210a. The barrier 1210 defines a fluid reservoir 1240, which may be coated with a hydrophobic material. In the illustrated embodiment, the fluid reservoir 1240 is made of a wicking material. The chamber 1210 may be made of, for example, acrylic. The device 1200 further includes a wicking component 1230 that transports fluid from the reservoir 1240 to an analyte sensor 1220. Although not shown, a diffusion-limiting material such as one described above acts as a barrier between the wicking component 1230 and the sensor 1220. At least a portion of the wicking component 1230 is adjacent to the opening 1210a of the chamber 1210. The reservoir 1240 is designed to receive a flow of biofluid, such as sweat, from the skin.

Over time, the biofluid fills the reservoir 1240 creating a pressure that forces fluid to begin moving through the opening 1210a (FIG. 12B). There may be a hydrophobic medium (e.g., air) between the wicking component 1230 and the opening 1210a. The fluid pressure from the fluid in the reservoir and the hydrophobicity of the surrounding medium causes the water to bulge out of the opening 1210a. As shown in FIG. 12C, due to capillary forces, the fluid moving through the opening 1210a contacts the wicking component 1230 before spilling out over the top of the chamber 1210. The distance between the wicking component 1230 and the opening 1210a is determined based on, for example, the properties of the sample fluid and the size of the opening 1210a. As the sample of the biofluid travels through the wicking component, the biofluid in the reservoir 1240 loses contact with the wicking component 1230 (FIG. 12D). As more biofluid enters the reservoir 1240, the process is repeated. Thus, the wicking component 1230 transports a series of discrete, quantized samples of the biofluid across the analyte sensor 1220. As with the device 1100, the measured current will increase rapidly and then decrease as the analyte in each sample is consumed by the sensor 1220 or as it flows away from the sensor 1220.

With reference to FIG. 13, the formation and final volume of the droplet are controlled at least in part by the height (h) of the wicking component 1230 relative to the opening 1210a and the diameter (D) thereof. To understand the amount of volume that could be dispensed, the droplet roughly estimated as the volume of a hemisphere (V=⅔π(h)³) (assuming the height and opening are the same size, viz. D=h), the volume could be as small as 1 nL (e.g., for h=0.8 mm) or several hundred of μL's (e.g., for h>4 mm). To dispense a series of discrete droplets, the capillary bridge (shown in FIG. 11B) must repeatedly break. For example, the capillary bridge may repeatedly break if 1) the hydrophobicity properties do not change and 2) there is enough input flow resistance to allow a droplet to break away, which can be controlled, for example, by making the supply channel thinner and longer than the droplet chamber or by making h larger than D. Decreasing or increasing either h or D will directly affect the volume of the dispensed droplet and frequency of the droplets entering the wicking component 1230. Thus, the volume of the droplets and frequency of the sensing may be determined by varying h or D. In addition, increasing the hydrophobicity or structure of the chamber 1210 and/or opening 1210a may also affect the formation and volume of the droplet. For example, the chamber 1210 includes a sharp rim around the opening 1210a that increases pinning of the droplet.

What is claimed is:

1. A device for sensing a biofluid that is adapted to be placed on skin, comprising:
at least one analyte-consuming sensor for measuring at least a first analyte concentration of an analyte in a first biofluid sample having a first biofluid sample flow rate; and
at least one additional component that maintains analyte-consuming sensor measurements within 20% of the first concentration measurement when a biofluid sample flow rate is less than or equal to 2 times the first biofluid sample flow rate.

2. The device of claim 1, where the additional component is a concentration regulating component located between the biofluid and said analyte-consuming sensor, wherein said concentration regulating component is adapted to be in contact with an advective flow of the biofluid.

3. The device of claim 2, wherein said concentration regulating component is a diffusion limiting material.

4. The device of claim 3, wherein said diffusion limiting material is adapted to allow passage of the first analyte and prevent passage of the biofluid.

5. The device of claim 1, further comprising:
at least one secondary sensor.

6. The device of claim 5, wherein the at least one secondary sensor is at least one of the following: a second analyte-specific sensor for sensing the first analyte; a pH sensor; a galvanic skin response (GSR) sensor; a sample generation rate sensor; a micro-thermal flow sensor; a sweat conductivity sensor; a skin impedance sensor; or an ion selective electrode sensor.

7. The device of claim 5, wherein the at least one secondary sensor is adapted to be in contact with an advective flow of biofluid.

8. The device of claim 1, wherein the device uses an algorithm to correlate at least one of the following measurements of the biofluid sample with an analyte concentration: a measurement by the analyte-consuming sensor; a pH measurement; a flow rate measurement; or a salinity measurement.

9. The device of claim 1, wherein the device uses a data table to correlate at least one of the following measurements of the biofluid sample with an analyte concentration: a measurement by the analyte-consuming sensor; a pH measurement; a flow rate measurement; or a salinity measurement.

10. The device of claim 1, further comprising:
an analyte-sensing channel; and
a plurality of analyte-consuming sensors arranged along said analyte-sensing channel, wherein the device is configured to determine a biofluid sample flow rate based on a comparison of outputs from the plurality of analyte-consuming sensors.

11. The device of claim 1, further comprising a catalyst region containing a catalyst, where the catalyst region is in fluid communication with the analyte-consuming sensor.

12. The device of claim 11, wherein the catalyst region is a coating on the analyte-consuming sensor.

13. The device of claim 11, wherein the catalyst is an enzyme.

14. The device of claim 13, wherein the enzyme is one of the following: a dehydrogenase; an oxidase; a deglycosylated enzyme; an RNAzyme; a DNAzyme; or a polymeric matrix.

15. The device of claim 1, wherein the analyte-consuming sensor is configured to indirectly measure pH by measuring a redox of protons.

16. The device of claim 1, wherein the analyte-consuming sensor is configured to measure a redox active metabolite.

17. The device of claim 1, wherein the analyte-consuming sensor is configured to indirectly measure a redox active metabolite.

18. The device of claim 1, further comprising:
an analyte-sensing channel having a known fluid volume;
a plurality of analyte-consuming sensors; and
at least one catalyst region.

19. The device of claim 18, where at least one catalyst region is located upstream of each of the plurality of analyte-consuming sensors relative to a flow direction of the biofluid.

20. The device of claim 18, wherein a plurality of paired catalyst regions and sensors are arranged along the analyte-sensing channel in the flow direction of the biofluid.

21. The device of claim 18, wherein there are a plurality of catalyst regions each of which is co-located with one of the plurality of analyte-consuming sensors, so that the biofluid must first react with one of the catalyst regions before reaching one of the analyte-consuming sensors.

22. The device of claim 21, wherein each of the catalyst regions are in one of the following arrangements: coating a surface of each of the analyte-consuming sensors or suspended in an immobilization matrix.

23. A method for sensing a biofluid using a device adapted to be placed on skin, comprising:
measuring a first analyte concentration of an analyte in a first biofluid sample having a first biofluid sample flow rate using an analyte-consuming sensor;
measuring the first biofluid sample flow rate; and
maintaining a subsequent analyte concentration within 20% of the first analyte concentration when a subsequently measured biofluid flow rate is less than or equal to 2 times the first biofluid sample flow rate.

24. The method of claim 23, wherein maintaining the subsequent analyte concentration includes using a concentration regulating component between the biofluid and said analyte-consuming sensor, the concentration regulating component being in contact with an advective flow of the biofluid.

25. The method of claim 23, further comprising:
correlating at least one of the following measurements of the biofluid sample with the first analyte concentration or a subsequently measured analyte concentration: a measurement by the analyte-consuming sensor; a pH measurement; a flow rate measurement; and or a salinity measurement.

26. The method of claim 23, wherein the device comprises an analyte-sensing channel and a plurality of analyte-consuming sensors arranged along said analyte-sensing channel, the method further comprising:
determining a biofluid sample flow rate based on a comparison of outputs from the plurality of analyte-consuming sensors.

27. The method of claim 23, further comprising:
converting the analyte in the biofluid to a component that is measurable by the analyte-consuming sensor using a catalyst.

28. The method of claim 23, wherein measuring the first analyte concentration includes applying electrical pulses to the analyte-consuming sensor.

29. The method of claim 28, further comprising:
adjusting a duration of the electrical pulses based on changes in the biofluid flow rate.

30. The method of claim 23, further comprising:
delivering the first biofluid sample and subsequent biofluid samples to the analyte-consuming sensor, each biofluid sample having a discrete volume.

31. The method of claim 30, further comprising:
forming the first biofluid sample and subsequent biofluid samples based at least in part on capillary forces.
